(19) United States
(12) Patent Application Publication
(10) Pub. No.: US 2020/0268657 A1
(43) Pub. Date: Aug. 27, 2020

(54) SUPPLEMENT TABLET AND PACKAGING

(71) Applicants: Michael Gulyas, Laguna Beach, CA (US); Tab Protein, LLC, Trumbull, CT (US)

(72) Inventor: Michael Gulyas, Laguna Beach, CA (US)

(21) Appl. No.: 16/630,647

(22) PCT Filed: Jul. 13, 2018
(86) PCT No.: PCT/US2018/042180

§ 371 Date: Jan. 13, 2020

Related U.S. Application Data
(60) Provisional application No. 62/532,142, filed on Jul. 13, 2017.

(51) Int. Cl. A61K 9/00; A61K 31/352; A61K 9/20; A61J 1/03; A61K 9/28; A23L 33/105; A23L 33/17

(52) U.S. Cl. CPC A61K 9/0095; A61K 31/352; A61K 9/2063; A23L 33/17; A61K 9/282; A23L 33/105; A61J 1/035

(57) ABSTRACT

A single use packaged product including a tablet containing supplement powder with a density such that the tablet in will break apart to form a shake when submerged and manually shaken in a container of liquid. The packaged product also includes a package having a first portion and a second portion together defining a receiving area. The tablet is disposed inside of the receiving area, and enclosed by the package for protection from contamination and accidental exposure to moisture. The package is configured to expel the tablet via manual manipulation of the package.

### CROSS-REFERENCE TO RELATED APPLICATIONS

This application claims the benefit of U.S. Provisional Patent Application 62/532,142, filed on Jul. 13, 2017, which is hereby incorporated by reference in its entirety.

The present applicant requests that the present international application be processed according to the Patent Cooperation Treaty.

### TECHNICAL FIELD

The present disclosure relates generally to a supplement tablet and packaging therefor, and more particularly to a tablet containing a supplement or additive that breaks apart in water and is packaged in an easy-open and protective packaging unit.

### BACKGROUND

There is a growing need for faster and more efficient mechanisms to distribute vitamins, supplements, and pharmaceutical substances to the average person in their daily life.

One example of an industry where distribution and transport is particularly troublesome is in the supplement industry, which serves a vast number of people from body-builders to vegans/vegetarians. Similar problems are faced within markets associated with trade in pharmaceuticals and additives. People consume a wide variety of supplements and additives for a variety of reasons, and these industries have continued to grow in diversity in terms of the supplements and additives offered as well as the types of consumers being targeted. One of the challenges faced by these industries is how to package and distribute supplements, pharmaceuticals and additives so that they can be easily carried and consumed at a convenient time by the consumer.

There is a pervasive trend in the supplement industry, and particularly in the protein powder industry, to sell the supplement in a loose powder form packaged inside of a tub with a scooper. The scooper is generally kept inside the tub, and in order to consume the powder a consumer will generally open the tub, scoop out the desired amount of supplement powder into a container, and then mix the supplement powder with water until it forms a suspension and can be consumed. This process is not only time consuming, but requires that the user know the appropriate amount of protein that needs to be consumed. The biggest problem with the current packaging and distribution process is that the tubs commonly used to store and sell the supplement powder are not convenient for carrying around, e.g. to take to the gym or carry with you to work, etc. Moreover, such delivery mechanisms do not allow for easy insertion into the necks of plastic water bottles and other beverages.

In addition, it is unhygienic for a consumer to reach a scooper into the tub with uncovered hands, especially if the consumer is sweaty after a workout. There is also a lack of precision and control over the quantity of protein included in each shake due to the fact that the scooper is of a uniform size and there is no convenient or accurate way to measure out a fraction of a scoop. If multiple supplements are to be included in a shake, the process of opening the tub, measuring the powder, pouring it into the container, replacing the scooper, and closing the tub must be performed for each supplement.

Additionally, a need exists for the efficient delivery of additives such as cannabis derivatives. Existing delivery mechanisms, including edible food-based delivery systems and smoking mechanisms remain inefficient and associated with undesirable side effects. Such delivery mechanisms also are accompanied by undesirable pollutants. Cannabis derivatives and cannaboids often are mis-measured and mis-dosed. These undesirable aspects of existing cannabinoid delivery mechanisms remain particularly problematic in association with the delivery of cannaboids in association with medical usage. This is particularly the case with regard to THC-A, which has been found to have substantial benefit to the peripheral nervous system (for pain management), the gut and the immune system. A need exists for an improved delivery system that better delivers the more easily extracted THC-A, which is water-soluble and does not require heat to extract, and therefore more easily retains other beneficial compounds such as terpenes and CBD. Aspects of the present invention are directed to these and other problems.

### SUMMARY

According to an aspect of the present invention, a single use packaged product includes a tablet containing supplement powder with a density such that the tablet will break apart to form a shake when submerged and manually shaken in a container of liquid, the supplement powder including protein. In alternative embodiments, the phrase “supplement powder” as used herein refers to a powder comprising a variety of additives, optionally including cannabis, cannabis derivatives, and more specifically Tetrahydrocannabinol (THC), THC-A, and/or Cannabidiol (CBD). The packaged product also includes a package having a first portion and a second portion together defining a receiving area. The tablet is disposed inside of the receiving area, and enclosed by the package for protection from contamination and accidental exposure to moisture, and the package is configured to expel the tablet via manual manipulation of the package.

According to another aspect of the present invention, a tablet includes supplement powder with a density such that the tablet will break apart to form a viscous consumable fluid, such as a shake, when submerged and manually shaken in a container of liquid. The supplement powder includes protein powder. In an alternative embodiment, the supplement powder includes cannabis or a cannabis product, such as THC or THC-A. Aspects of the invention incorporate source chemistry and pressed pill baselines that provide for accurate payload delivery in solution and reproducibility. Moreover, aspects of varying embodiments of the invention are tailored to allow for optimal dissolution time, dissolution in hot and cold liquids, in different types of liquids, and in the dissolution and delivery of multiple doses.

According to another aspect of the present invention, a supplement distribution and packaging system includes a consumer computing device for inputting supplement selections and consumer preferences, and a distributor computer for receiving the supplement selections and the consumer preferences, the distributor computer having a supplement selection program executing thereon for generating a supplement order based on the supplement selections and the consumer preferences. The system also includes a tablet maker for receiving the supplement order and generating tablets by compressing a supplement powder, where a composition of the supplement powder is determined according to the supplement order, and the supplement tablets are compressed to a density such that the tablet will break apart to form a shake when submerged and manually shaken in a container of liquid. The system also includes a packager for receiving the tablets and supplement order and packaging the tablets based thereon to produce a packaged product for consumption.

In addition to, or as an alternative to, one or more of the features described above, further aspects of the present invention can include one or more of the following features, individually or in combination: the package is sized to fit in a clothing pocket; multiple ones of the package are in a sheet; multiple ones of the package are in a box; a second tablet not having protein is in the package; the supplement powder can be pre-selected by a customer; the first portion is removable, and is configured to be peeled off of the second portion to expose the tablet in the receiving area; the first portion is adhesively bonded to the second portion; the first portion is breakable, and is configured to be broken such that the tablet may be expelled from the receiving area through the break in the first portion; the first portion is a sheet of material that is malleable and has low shear strength; the tablet has a coating for dimensional stability; the package is readily transportable for use away from home; the supplement selections include information on at least one of a protein type, a protein flavor, a cannabis type, a cannabis flavor, a protein quantity, a Tetrahydrocannabinol (THC) quantity, a THC-A quantity, vitamins, minerals, and nutrients that a consumer wishes to be included in their tablets; the consumer preferences include information about the consumer's age, weight, sex, build, training goals, medical treatment needs, and dietary restrictions, and the supplement selection program determines the supplement selections for a consumer based on the consumer preferences entered by the consumer; the tablet forms a suspension when mixed with water; the coating contains protein and/or THC.

These and other aspects of the present invention will become apparent in light of the drawings and detailed description provided below.

### BRIEF DESCRIPTION OF THE DRAWINGS

The above and other aspects, features and advantages of certain embodiments of the present invention will be more apparent from the following detailed description taken in conjunction with the accompanying drawings, in which:

FIG. 1 illustrates a front plan view of a supplement tablet and packaging according to the present disclosure, with the tablet being expelled from the package into a container with liquid.

FIG. 2 illustrates a front plan view of the supplement tablet and packaging of FIG. 1 with several packages in a sheet.

FIG. 3 illustrates a bottom plan view of the supplement tablet and packaging of FIG. 1 having a removable portion.

FIG. 4 is a block diagram of a system for customizing the supplement tablet and packaging of FIG. 1.

While the invention is described with reference to the above drawings, the drawings are intended to be illustrative, and the invention contemplates other embodiments within the spirit of the invention.

### DETAILED DESCRIPTION

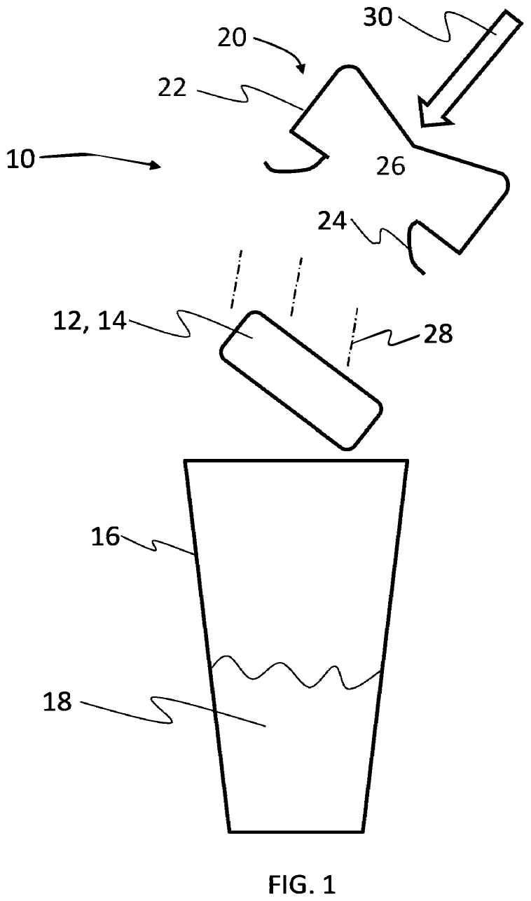

Referring to FIG. 1, the present disclosure describes a single use packaged product 10 including a tablet 12 containing supplement powder 14. In an embodiment, the tablet 12 is substantially round and of a diameter such that it can fit within a standard sized plastic water bottle having an opening of a diameter no greater than two inches. In an embodiment, the diameter of the tablet 12 is approximately 1 inch. The density of the supplement powder 14 is such that the tablet 12 will break apart to form a viscous consumable fluid, such as a shake (i.e. a “protein shake”, as in the drinks commonly consumed by people before/after weight training, and which commonly contain ingredients other than just protein; or a “cannabis shake”) when submerged and manually shaken in a container 16 of liquid 18. The supplement powder 14 in an embodiment of the invention includes a protein. Particularly in embodiments where the tablet 12 comprises supplement powder 14 that includes a protein, the tablet 12 is much larger, having a diameter of up to 3 inches in size. In alternative embodiments, particularly in embodiments wherein the supplement powder 14 includes a protein, the size of the tablet 12 is even larger, up to 6 inches in diameter and up to 3 inches in height. In an alternative embodiment, the supplement powder 14 in an embodiment of the invention includes a cannabis product. In an embodiment of the invention, the cannabis product comprises THC. In an embodiment of the invention, the cannabis product comprises THC-A. In various embodiments, the tablet 12 incorporates a matrix, which as one skilled in the art recognizes is the middle section or core of the tablet 12. In an embodiment, the matrix is comprised of the supplement powder 14.

Still referring to FIG. 1, the packaged product 10 also includes a package 20 having a first portion 22 and a second portion 24, which together defining a receiving area 26. The tablet 12 is initially disposed inside the receiving area 26, and enclosed by the package 20 for protection from contamination and accidental exposure to moisture. The package 20 is configured to expel the tablet 12 as indicated by motion lines 28, via manual manipulation of the package 20 as indicated by arrow 30.

In some embodiments, the manual manipulation is provided by a person depressing the center of the package 20 with their finger in order to push the tablet against the second portion and thereby cause it to break, in order for the tablet to be released from the package 20 and fall into the container 16 holding the liquid 18. The rounded side of the first portion 22 is structurally rigid so that the package 20 can withstand a compressive force sandwiching the entire package 20 without deformation of the package 20, thereby protecting the tablet 12 from being crushed. The flat side of the first portion 22, which is structurally flexible so that it can be pushed in to expel the tablet 12 with a relatively small force directed toward a central area of the flat side of the first portion 22. The flexible material is used so that the force (e.g. as indicated by arrow 30) on the central area of the flat side is substantially transferred to the tablet 12, and thereby substantially transferred to an interior side of the second portion 24. This causes the second portion 24 to break, releasing the tablet 12 from the package 20. The second portion 24 is a sheet of material that is malleable and has intentionally low shear strength, e.g. aluminum foil. The first portion is made from a material that is somewhat flexible and also generally water-resistant, e.g. a polymer.

In other embodiments, the tablet 12 has a coating on its outer surface. The coating adds dimensional stability and aids in the mixing of the tablet 12 with the liquid 18. The coating contains protein, and may also contain protein, vitamins, medicines, THC, THC-A, caffeine, and other agents or supplements that may be found in the supplement powder 14. In an embodiment, the coating serves as a moisture barrier. The present inventors recognize that to maintain optimal effervescent qualities, the matrix of the tablet should maintain optimized moisture levels in accordance with the teachings contained within U.S. Pat. No. 6,495,163 with a filing date of Sep. 23, 1998 which is hereby incorporated by reference in its entirety. The coating may be an effervescent coating to aid in the mixing of the tablet 12 with the liquid 18, or the tablet 12 itself may be an effervescent tablet due the incorporation of effervescent substances within its composition. In such embodiments, the effervescence may be accomplished through the incorporation of sodium bicarbonate, and/or potassium bicarbonate in the composition of supplement powder 14 and/or in the coating of the tablet. In an alternative embodiment, the effervescence may be accomplished through use of arginine in the supplement powder 14 and/or in the coating of the tablet. The present inventors have recognized the benefit of using arginine as a non-sodium alternative to provide effervescence, as many seek to avoid the addition of sodium to their diets. These ingredients react with water to create an acid base reaction releasing CO2 and aiding in the breakup of the tablet 12. As used herein, effervescent means that the substance (in either the coating and/or supplement powder 14) reacts with a liquid causing the release of gas. Moreover, in an embodiment, the tablet 12 may incorporate one or more disintegrants. Disintegrants in the context of the invention are described as substances that aid in the breakdown of the tablet 12 into a fluid. More precisely, disintegrants are described as substances intended to aid in the break up of the compacted mass of the tablet 12 when it is put into a fluid environment. Disintegrants promote moisture penetration and dispersion of the tablet matrix. In embodiments of the invention, particularly embodiments that incorporate protein in the supplement powder 14, the tablet 12 incorporates Chitosan as a disintegrant. The present inventors have recognized that Chitosan assists with the breakdown of the tablet 12 containing a supplement powder 14 comprising protein once it reaches a wet substance. In alternative embodiments, the tablet 14 may incorporate any of the following as a disintegrant: crosscarmellose sodium, Ac-Di-Sol, primellose, vivasol, crosdpovidone, kollidon, polyplasdone, sodium starch glycolate, alginic acid, satialgine, soy, polysaccharides, emcosoy, and/or calcium silicate. In an embodiment, the inclusion of one or more disintegrants causes rapid dispersion of the tablet 12 once placed in a liquid. In an embodiment of the invention, the breakup of the tablet within the fluid would take approximately 15-20 seconds to fully disperse within the fluid if the supplement powder 14 contains protein. In an embodiment where the supplement powder 14 contains a cannabis derivative, the time to dispersion would take approximately 10-15 seconds. While effervescent typically refers to the release of carbon dioxide, it can refer to other gases escaping as well. In embodiments of the invention, the supplement powder is configured to comprise a dosage of THC in an amount between 1 milligram and 10 milligrams. In embodiments of the invention, the supplement powder is configured to comprise an amount between 0.5 milligrams of CBD and 8 milligrams of CBD. In an embodiment of the invention, the supplement powder is configured to contain precisely 5 milligrams of THC and 2 milligrams of CBD. The present inventors have recognized that THC-A provides many of the desired physiological effects of THC, however, due to being acidic and water-soluable, THC-A does not cross the blood brain barrier and therefore does not create undesirable psychological effects. In varying embodiments of the invention, therefore, within the supplement powder, THC-A is used in place of THC.

In yet other embodiments, there is a second tablet 12 included in the receiving area 26 that does not have protein in it. In yet alternative embodiments, there is a second tablet 12 included in the receiving area 26 that does not have cannabis, a cannabinoid, THC, or THC-A in it. The second tablet 12 would have one or more alternative supplements selected by the consumer. In other embodiments, a third tablet 12 (or multiple additional tablets 12) may be included in the receiving area 26. For embodiments having more than one tablet 12, an advantage is provided in that the consumer can see each supplement that they are taking in a separate tablet form, while still being able to store and consume them all at once. In other embodiments, multiple tablets 12 are used to aid in the speed of mixing the tablet 12 with the liquid 18, and the multiple tablets 12 do not necessarily have a different composition.

In other embodiments, the shake formed by mixing the tablet 12 with the liquid 18 is a suspension where the supplement powder 14 is suspended in water. In yet other embodiments, the supplement powder 14 forms a solution when mixed with water. The tablet 12 is designed to break apart in water relatively quickly, for example after being manually shaken in a container 16 of liquid 18 for a period of time in the range of 1 second to 60 seconds.

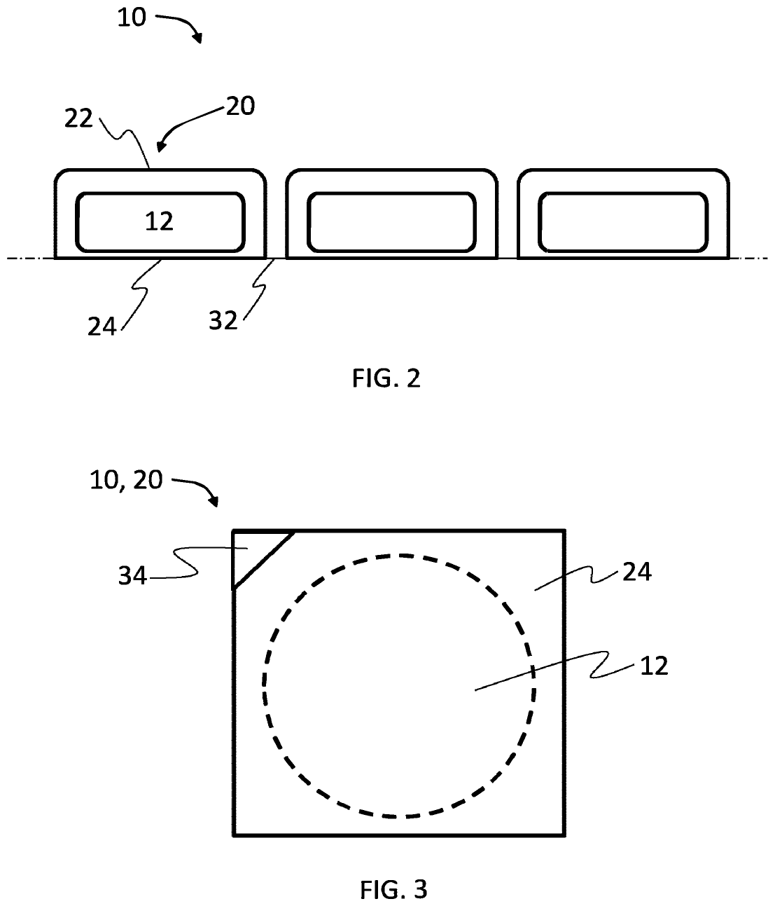

Referring to FIG. 2, the packaged product 10 is shown with the tablet 12 contained in the package 20. The first portion 22 of the package 20 is attached to the second portion 24. A sheet 32 connects the package 20 to other packages 20 such that multiple packages 20 can be transported easily. In other embodiments, the sheet 32 connects the first portion 22 of the package 20 to the first portion 22 of a neighboring package 20. The sheet 32 may be designed such that the packages 20 are all aligned in one row, or it may be designed such that the packages are in a 2×2 square (i.e. 2 packages 20 across and 2 packages 20 down), or 2×3, or 2×4 rectangle, or other configurations known by those of skill in the art familiar with the present disclosure. In other embodiments, the package 20 is a stand-alone item which is distributed with other packages 20 in a box or a tub, and is not connected to the other packages 20 in the box.

In some embodiments, the receiving area 26 is shaped to receive the tablet 12 such that a ratio of a receiving area height to a receiving area width is approximately equal to a ratio of a tablet height to a tablet width. The package 20 is cylindrically shaped, and the rounded side of the first portion 22 and the first flat side of first portion 22 defines the receiving area 26, and the second portion 24 is a removable portion or a breakable portion.

Referring to FIG. 3, the packaged product 10 is shown with the dotted line indicating the position of the tablet 12 behind the second portion 24 of the package 20. The second portion 24 has a tab 34 which is designed to help with the removal of the second portion 24 from the first portion 22 in order to expose the tablet 12 just prior to removal from inside the package 20. The tab 34 is designed as a corner of the second portion 24 that is not adhesively connected to the first portion 22, thus allowing a person to grip the tab 34 and peel the second portion 24 off of the first portion 22. This design for opening the package via manual manipulation is an alternative to the method described above (and as shown in FIG. 1.) where the tablet 12 is “popped out” of the package 20 via a break in the second portion 24. In other embodiments, the package 20 is torn open, with one or both of the first portion 22 and second portion 24 being tearable. In yet other embodiments, the first portion 22 has a form fit with the second portion 24 and the two parts can be popped apart to release the tablet 12. Other variations of opening the package 20 are known by those of skill in the art having the benefit of the present disclosure.

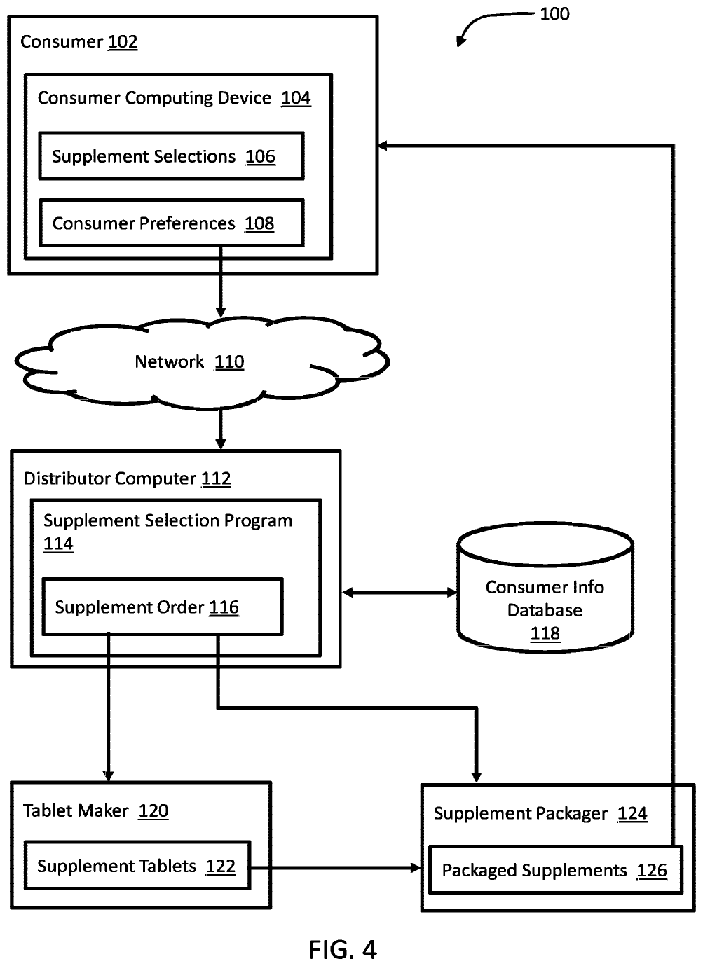

Referring to FIG. 4, a block diagram of a system 100 for customizing the packaged product 10 is shown. The system 100 includes a consumer 102, which operates a consumer computing device 104 to input supplement selections 106 and consumer preferences 108. The supplement selections 106 and consumer preferences 108 are sent to a distributor computer 112 through a network 110. The distributor computer 112 has a supplement selection program 114 executing thereon for generating a supplement order 116 using the information provided in the supplement selections 106 and the consumer preferences 108. The distributor computer 112 may store information such as the supplement selections 106, consumer preferences, or supplement order 116 on a consumer information database 118 for later usage.

Still referring to FIG. 4, a tablet maker 120 receives the supplement order 116 and uses it to form supplement tablets 122 having the right supplements, e.g. supplement type, quantity, flavor, etc. The supplement tablets 122 are compressed by the tablet maker 120 to an appropriate density so that they will break apart when shaken in water, but not break apart during typical handling and transport. The supplement tablets are received by the supplement packager 124 which packages the tablets 122 to form packaged supplements 126, which are then sent to the consumer 102.

In some embodiments, the supplement selections 106 may include information such as protein type, protein flavor, cannabis type, cannabis flavor, protein quantity, cannabis quantity, THC quantity, THC-A quantity, vitamins, minerals, and nutrients that a consumer 102 wishes to be included in the packaged supplements 126 that are sent to them. In other embodiments, the consumer preferences 108 include information about the consumer's age, weight, sex, build, training goals, medical needs, and dietary restrictions, and the supplement selection program 114 determines the supplement selections 106 for a consumer 102 based on the consumer preferences 108 entered by the consumer 102. This embodiment allows the consumer 102 to receive a product tailored to their needs even if they do not have the expertise to make the selections on their own. In other embodiments, the consumer 102 can include their prescribed pharmaceutical drugs, allergy medication, daily vitamins, medical or recreational marijuana, and other substances with their supplement selections 106 so that they are given an all-inclusive tablet that meets their dietary needs. An advantage of this embodiment is that certain substances may be absorbed by the body faster when consumed in the form of a liquid solution or suspension.

The packaged product 10 offers several advantages over known products. Among other things, the packaged product 10 is designed for a single use, allowing a consumer to easily transport and consume their protein powder in the form of a tablet so that when the consumer wishes to make a protein shake, all they have to do is expel the tablet 12 from the package 20 and shake it up with water to create the shake. This allows, for instance, gym-goers to avoid having to use a scooper with a tub of protein which is bulky and inconvenient to carry around, and also requires measuring, which can be inconvenient and unhygienic. The package 20 also provides an advantage by being sized to fit in a clothing pocket or a purse, e.g. the pocket of athletic wear such as a small thigh pocket on running spandex designed for a key (i.e. a “key pocket”) or small zipper pocket in a fleece for storing a credit card (i.e. a “card pocket”). In other examples, the package 20 offers the advantage of being sized for concealment.

The packaged product system 100 also allows for increased efficiency since a consumer may customize their single use packaged product so that each tablet 12 has precisely the right supplements for the dietary needs. For example, an athlete taking multiple supplements seeking to control their supplement intake with precision and ease can benefit greatly from the packaged product system 100 since it replaces a series of complicated and time-consuming measurements of various powders and pills from multiple containers each time the athlete wishes to consume a supplement shake. Such consumption might also take place by anyone, athlete or otherwise, who wishes to improve their overall wellness. An athlete just starting out with a supplement regiment can also benefit from the packaged product system since it can aid a consume in selecting the supplements they wish to take, or even calculate certain things for them, e.g. determine quantity based on their training goals/weight/sex, or e.g. determine protein type (plant or casein) based on dietary restrictions or allergies.

The packaged product 10 also provides an advantage since it may be used to deliver other ingestible items or combined with a user's other vitamins/medicine to meet the user's supplement/medical needs with one item. Advantages also include that large doses of ingredients can be taken in a single serving, and nutrients can be absorbed by the body faster than if taken in other forms such as pills.

With conventional solid tablets or capsules that are swallowed by a user, the tablet is transported to the user's stomach after being swallowed, where it dissolves gradually. The passage time varies in different people, depending on anatomical and physiological factors. In case of a longer passage time, solid dosage forms can dissolve partially and this may cause irritation of mucous membranes. Advantages of the packaged product 10 include that because it is dissolved in liquid the ingredients are evenly distributed in the solution, and high, localized concentrations cannot occur.

While several embodiments have been disclosed, it will be apparent to those of ordinary skill in the art that aspects of the present invention include many more embodiments and implementations. Accordingly, aspects of the present invention are not to be restricted except in light of the attached claims and their equivalents. It will also be apparent to those of ordinary skill in the art that variations and modifications can be made without departing from the true scope of the present disclosure. For example, in some instances, one or more features disclosed in connection with one embodiment can be used alone or in combination with one or more features of one or more other embodiments.

What is claimed is:

1. A single use packaged product, comprising:
a tablet containing supplement powder with a density such that the tablet will break apart to form a shake when submerged and manually shaken in a container of liquid;
a package having a first portion and a second portion together defining a receiving area;
the tablet is disposed inside of the receiving area, and enclosed by the package for protection from contamination and accidental exposure to moisture; and
the package is configured to expel the tablet via manual manipulation of the package.

2. The single use packaged product of claim 1, wherein the package is sized to fit in a clothing pocket.

3. The single use packaged product of claim 1, wherein multiple ones of the package are in a sheet.

4. The single use packaged product of claim 1, wherein multiple ones of the package are in a box.

5. The single use packaged product of claim 1, wherein a second tablet not having protein is in the package.

6. The single use packaged product of claim 1, wherein the supplement powder can be pre-selected by a customer.

7. The single use packaged product of claim 1, wherein the first portion is removable, and is configured to be peeled off of the second portion to expose the tablet in the receiving area.

8. The single use packaged product of claim 7, wherein the first portion is adhesively bonded to the second portion.

9. The single use packaged product of claim 1, wherein the first portion is breakable, and is configured to be broken such that the tablet may be expelled from the receiving area through the break in the first portion.

10. The single use packaged product of claim 8, wherein the first portion is a sheet of material that is malleable and has low shear strength.

11. The single use packaged product of claim 1, wherein the tablet has a coating for dimensional stability.

12. The single use packaged product of claim 11, the coating comprising an arginine.

13. The single use packaged product of claim 1, wherein the package is readily transportable for use away from home.

14. The single use packaged product of claim 1, the supplement powder comprising protein.

15. The single use packaged product of claim 1, the supplement powder comprising cannabis.

16. The single use packaged product of claim 1, the supplement powder comprising THC.

17. A tablet, comprising:
supplement powder with a density such that the tablet will break apart to form a shake when submerged and manually shaken in a container of liquid.

18. The tablet of claim 17, the supplement powder comprising arginine.

19. The tablet of claim 17, the supplement powder comprising protein.

20. The tablet of claim 17, the supplement powder comprising cannabis.

21. The tablet of claim 17, the supplement powder comprising THC.

22. A supplement distribution and packaging system, comprising:
a consumer computing device for inputting supplement selections and consumer preferences;
a distributor computer for receiving the supplement selections and the consumer preferences, the distributor computer having a supplement selection program executing thereon for generating a supplement order based on the supplement selections and the consumer preferences;
a tablet maker for receiving the supplement order and generating tablets by compressing a supplement powder, where a composition of the supplement powder is determined according to the supplement order, and the supplement tablets are compressed to a density such that the tablet will break apart to form a shake when submerged and manually shaken in a container of liquid; and
a packager for receiving the tablets and supplement order and packaging the tablets based thereon to produce a packaged product for consumption.

23. The supplement distribution and packaging system of claim 22, wherein the supplement selections include information on at least one of a protein type, a protein flavor, a protein quantity, vitamins, minerals, and nutrients that a consumer wishes to be included in their tablets.

24. The supplement distribution and packaging system of claim 22, wherein the consumer preferences include information about the consumer's age, weight, sex, build, training goals, and dietary restrictions; and the supplement selection program determines the supplement selections for a consumer based on the consumer preferences entered by the consumer.

25. The supplement distribution and packaging system of claim 22, wherein the tablet forms a suspension when mixed with water.

26. The supplement distribution and packaging system of claim 22, wherein the coating contains protein.

27. The supplement distribution and packaging system of claim 22, wherein the coating contains cannabis.

28. The supplement distribution and packaging system of claim 22, wherein the coating contains THC-A.
